(19) United States
(12) Patent Application Publication
(10) Pub. No.: US 2012/0276560 A1
(43) Pub. Date: Nov. 1, 2012

(54) BIOMARKERS FOR ASSESSING SIALIC ACID DEFICIENCIES

(76) Inventors: Emil D. Kakkis, Novato, CA (US); Daniel K. Darvish, Reseda, CA (US); Yadira Valles-Ayoub, Reseda, CA (US)

(21) Appl. No.: 13/329,036

(22) Filed: Dec. 16, 2011

Related U.S. Application Data
(60) Provisional application No. 61/483,031, filed on May 5, 2011.
(60) Provisional application No. 61/424,590, filed on Dec. 17, 2010.

(51) Int. Cl. G01N 33/566; G01N 33/577

(52) U.S. Cl. 435/7.92

(57) ABSTRACT

The present invention relates to methods of diagnosing, monitoring and assessing conditions of sialic acid deficiency such as Hereditary Inclusion Body Myopathy (HIBM) and to methods of predicting/determining responsiveness to treatment.

### CROSS REFERENCE TO RELATED APPLICATIONS

This application claims the benefit under 35 U.S.C. §119(e) of U.S. Application No. 61/424,590, filed 17 Dec. 2010; and U.S. Application No. 61/483,031, filed 5 May 2011, each of which is incorporated by reference in its entirety.

### TECHNICAL FIELD

Embodiments of the present invention relate to methods for determining the sialylation state of a polysialic acid-glycoprotein in a blood sample from a subject, and related methods for diagnosing, evaluating and managing conditions of sialic acid deficiency, such as Hereditary Inclusion Body Myopathy (HIBM).

### BACKGROUND

Sialic acid is the only sugar that contains a net negative charge and is typically found on terminating branches of N-glycans, O-glycans, and glycosphingolipids (gangliosides) (and occasionally capping side chains of GPI anchors). The sialic acid modification of cell surface molecules is crucial for many biological phenomena including protein structure and stability, regulation of cell adhesion, and signal transduction. Sialic acid deficiency disorders such as Hereditary Inclusion Body Myopathy (HIBM or HIBM type 2), Nonaka myopathy, and Distal Myopathy with Rimmed Vacuoles (DMRV) are clinical diseases resulting from a reduction in sialic acid production.

HIBM is a rare autosomal recessive neuromuscular disorder case by a specific biosynthetic defect in the sialic acid synthesis pathway. Eisenberg et al., Nat. Genet. 29:83-87 (2001). The disease manifests between the ages of 20 to 40 with foot drop and slowly progressive muscle weakness and atrophy. Patients may suffer difficulties walking with foot drop, gripping and using their hands, and normal body functions like swallowing. Histologically, it is associated with muscle fiber degeneration and formation of vacuoles containing 15-18 nm tubulofilaments that immunoreact like β-amyloid, ubiquitin, prion protein and other amyloid-related proteins. Askanas et al., Curr Opin Rheumatol. 10:530-542 (1998). Both the progressive weakness and histological changes initially spare the quadriceps and certain other muscles of the face. However, the disease is relentlessly progressive with patients becoming incapacitated and wheelchair-confined within one to two decades. There are no treatments currently available.

The causative mutations were identified for HIBM in the gene GNE, which encodes the bifunctional enzyme UDP-N-acetylglucosamine-2-epimerase/N-acetylmannosamine kinase (GNE/MNK). Studies of an Iranian-Jewish genetic isolate mapped the mutation associated with HIBM to chromosome 9p12-13. Argov et al., Neurology 60:1519-1523 (2003). Eisenberg et al., Nat. Genet. 29:83-87 (2001). DMRV is a Japanese variant, allelic to HIBM. Nishino et al., Neurology 59:1689-1693 (2002).

The biosynthesis steps and feedback regulation of GNE/MNK is depicted in FIG. 1. The production of sialic acid on glycoconjugates requires the conversion of N-acetylglucosamine (conjugated to its carrier nucleotide sugar UDP) to sialic acid. The sialic acid subsequently enters the nucleus where it is conjugated with its nucleotide sugar carrier CMP to make CMP-sialic acid, which is used as a donor sugar for glycosylation reactions in the cell. CMP-sialic acid is a known regulator of GNE/MNK activity. Jay et al., Gene Reg. & Sys. Biol. 3:181-190 (2009). Patients with HIBM have a deficiency in the production of sialic acid via the rate controlling enzyme GNE/MNK, which conducts the first two steps of this sequence: 1) epimerization of the glucosamine moiety to mannosamine with release of UDP, and 2) phosphorylation of the N-acetylmannosamine. The mutations causing HIBM occur in the regions encoding either the epimerase domain (GNE) or the kinase domain (MNK). Nearly twenty GNE mutations have been reported in HIBM patients from different ethnic backgrounds with founder effects among the Iranian Jews and Japanese. Broccolini et al., Hum. Mutat. 23:632 (2004). Most are missense mutations and result in decreased enzyme GNE activity and underproduction of sialic acid. Sparks et al., Glycobiology 15(11):1102-10 (2005); Penner et al., Biochemistry 45:2968-2977 (2006).

Knock-out of the Gne gene in mice is lethal as no sialic acid is incompatible with life, but knock-in introduction of human mutant forms of GNE/MNK have allowed the production of mouse models with human disease features. In the DMRV-HIBM mouse model in which Gne-deficient mice transgenically express the human GNE gene with D176V mutation (Gne^{-/-} hGNED176V-Tg), these mice show hypo-sialylation in various organs in addition to the characteristic features of muscle atrophy, weakness and degeneration, and amyloid deposition. In these mice, hypo-sialylation is documented from birth, yet the mice develop muscle symptoms only several weeks later, including decreased twitch force production in isolated muscles starting at 10 weeks of age and impairment of motor performance from 20 weeks of age onward. Muscle atrophy and weakness were, however, reduced or prevented after treatment with administration of a sialic acid metabolite, N-acetylmannosamine (ManNAc), sialic acid, or sialyl-lactose, in water. Malicdan et al., Nat. Medicine 15(6):690-695 (2009). All three sialic acid metabolites tested showed similar treatment effects. In another mouse model of HIBM in which knockin mice harbor the M712T Gne mutation, mice homozygous for the M712T Gne mutation died within 72 hours after birth, but lacked a muscle phenotype. Galeano et al., J. Clin. Investigation 117(6) 1585-1594 (2007). Homozygous mice, however, did have severe glomerular hematuria and podocytopathy, including effacement of the podocyte foot processes and segmental splitting of the glomerular basement membrane (GBM). Administration of ManNAc in water to mutant mice improved survival, improved renal histology including less flattened and fused podocyte foot processes, increased sialylation of renal podocalyxin, and increased sialylation of brain PSA-NCAM. Galeano et al., J. Clin. Investigation 117(6):1585-1594 (2007).

In individuals with DMRV, there is a 25% reduction of sialic acid in muscle tissue; however, there is no difference in sialic acid content in sera between DMRV individuals and normal control individuals. See Noguchi et al., JBC 279(12):11402-11407 (2004). Noguchi et al. reason that sialic acids are predominantly produced in the liver and transferred to synthesized glycoproteins, which are then released into the blood plasma. Free sialic acid in the plasma is derived from desialylation of these glycoproteins. GNE is expressed in the liver in large amounts; therefore, the reduction in enzymatic activity by mutations may not significantly affect the synthesis of sialic acid in the liver of DMRV patients, and sialic acid is present at concentrations comparable with normal blood levels. In contrast, Noguchi et al. reason that in DMRV skeletal muscles, the sialic acid contents are reduced. The reduced enzymatic activities along with weak expression of GNE protein are probably responsible for the more serious reduction in sialic acid synthesis in muscle tissue compared with plasma. Noguchi et al., JBC 279(12):11402-11407, 11406 (2004).

One sialic acid containing glycoprotein, Neural Cell Adhesion Molecule (PSA-NCAM) has been shown to play an important role in cell to cell interactions not only in brain, but also in muscle. Normally PSA-NCAM is sialylated with as many as 10 sialic acid residues per oligosaccharide chain in a structure referred to as poly sialic acid (PSA). PSA-NCAM is a component of the cell surface membrane of myoblasts in the muscle. It has been shown that HIBM patients have a form of PSA-NCAM on the surface of the muscle that is hypo-sialylated with reduced or completely absent sialic acid residues. Broccolini et al., Neurology 75 265-272 (2010). This has been confirmed in HIBM knock-in mice by showing these mice also produce PSA-NCAM that is hypo-sialylated. Gagiannis et al., Glycoconjugate Journal 24 125-130 (2007).

The current assessment of HIBM patients requires the use of a muscle biopsy and the assessment of sialylation of muscle bound glycoproteins such as PSA-NCAM. Ricci et al., Neurology, 66(5), 755-8 (2006); Broccolini et al., Neurology 75 265-272 (2010); Tajima et al., The American Journal of Pathology, 166(4) 1121-1130 (2005); Nemunaitis et al., J Gene Med, 12(5) 403-12 (2010). Muscle biopsies cannot be assessed regularly, are difficult to quantify and cannot be used reliably for regular management or drug development studies.

Given the problems associated with current methods for diagnosing HIBM and determining responsiveness to and/or monitoring treatment of HIBM patients, there is a need for methods which allow quantification of the biochemistry and easy detection of hypo-sialylated glycoproteins.

### BRIEF SUMMARY OF THE INVENTION

Embodiments of the present invention include methods for diagnosing a condition of sialic acid deficiency in a subject comprising determining the sialylation state of a polysialic acid-glycoprotein in a blood sample from the subject, and diagnosing the subject as sialic acid deficiency if the sialylation state is less than a pre-determined level. In some embodiments, the sialylation state of the polysialic acid-glycoprotein is determined based on the molecular weight of the polysialic acid-glycoprotein. In certain embodiments, the sialylation state is less than a pre-determined level if the molecular weight of the polysialic acid-glycoprotein in the blood sample is less than a pre-determined molecular weight. In certain embodiments, the polysialic acid-glycoprotein is expressed in muscle tissue.

In particular embodiments, the polysialic acid-glycoprotein comprises a polysialic acid polymer. In certain embodiments, the polysialic acid-glycoprotein comprises a polysialic acid polymer including from at least about 5 sialic acid residues to about 50 sialic acid residues. In specific embodiments, the polysialic acid-glycoprotein is polysialic acid-neural cell adhesion molecule (PSA-NCAM).

In certain embodiments, the blood sample is a serum or plasma sample. In some embodiments, the pre-determined level is a level determined based on a population without sialic acid deficiency.

Some methods further comprise recommending the subject for treatment of sialic acid deficiency. Certain methods further comprise determining the level of sialic acid deficiency based on the level of decrease of the sialylation state from the pre-determined level. In certain embodiments, the sialic acid deficiency is Hereditary Inclusion Body Myopathy (HIBM), Nonaka myopathy, or Distal Myopathy with Rimmed Vacuoles (DMRV).

Also included are methods for monitoring responsiveness or efficacy of a treatment to a subject suffering from sialic acid deficiency comprising determining the sialylation state of a polysialic acid-glycoprotein in a blood sample from the subject, wherein an increase of the sialylation state of the polysialic acid-glycoprotein is indicative of responsiveness or efficacy of the treatment. Some embodiments further comprise determining future treatment regimen based on the sialylation state of the polysialic acid-glycoprotein in the blood sample.

Particular embodiments include methods for determining whether a subject is suitable for a sialic acid replacement therapy comprising determining the sialylation state of a polysialic acid-glycoprotein in a blood sample from the subject, wherein a subject is suitable for a sialic acid replacement therapy if the sialylation state of the polysialic acid-glycoprotein is less than a predetermined level and wherein a subject is not suitable for a sialic acid replacement therapy if the sialylation state of the polysialic acid-glycoprotein is equal or higher than the pre-determined level.

Also included are methods for treating a subject comprising determining the sialylation state of a polysialic acid-glycoprotein in a blood sample from the subject, and administering a sialic acid replacement therapy to the subject if the sialylation state of the polysialic acid-glycoprotein is less than a pre-determined level.

Certain embodiments relate to one or more collections of molecular weight data comprising the molecular weight of a polysialic acid-glycoprotein in a blood sample from a testing subject. Some of these and related embodiments further comprise the molecular weight of the polysialic acid-glycoprotein in a blood sample from a control subject.

Some embodiments include methods for providing data comprising determining the sialylation state of a polysialic acid-glycoprotein in a blood sample from a subject, and providing the information of the sialylation state to a healthcare provider for diagnosing or treatment of the subject. Certain of these and related embodiments further comprise receiving the blood sample from the healthcare provider.

Also included are methods of assaying the sialylation state of a polysialic acid-glycoprotein in a subject comprising obtaining or receiving a blood sample of the subject, and determining the sialylation state of a polysialic acid-glycoprotein in the blood sample. In certain embodiments, the subject has, is at risk of having, or is suspected of having a condition of sialic acid deficiency.

### BRIEF DESCRIPTION OF THE DRAWINGS

FIG. 1 provides a diagram of intracellular sialic acid metabolism.

FIG. 2 show sialylated and non-sialylated NCAM from serum samples detected by a monoclonal anti-NCAM antibody. Standard size ladder 20-250 kD (lanes 1 and 10, Precision Plus Protein™ WesternC™ Standards, Bio-Rad, CA), two different HIBM patients (Lanes 2-3), two different patients suffering from non-GNE related myopathy (Lanes 4-5), normal human sera from two control individuals (Lanes 6-7), same normal sera as used in Lanes 6-7 following treatment with sialidase showing that upper and middle bands are lighter if sialic acid is removed (Lanes 8-9).

FIG. 3 shows sialylated and non-sialylated NCAM from serum samples detected by a monoclonal anti-NCAM antibody: molecular ladder (lanes 1 and 9), normal human serum from three healthy non-myopathic controls (lanes 2-4 and lanes 10-11), HIBM patients with GNE mutation (lane 5 and lane 12), myopathic patient without the HIBM GNE mutation (lane 6), serum from a HIBM patient on ManNAc self-treatment for 2 years (lane 7), sialidase treated, normal human serum from non-myopathic controls showing that the upper and middle bands disappear if sialic acid is removed (lane 8).

FIGS. 4A-4B show NCMA sialylation states in normal and HIBH human serum using the 123C3 antibody. FIG. 4A shows that without neuraminidase pre-treatment, PSA-NCAM from normal human serum appeared as a triplet ranging from about 110-130 kDa (lane 1). The triplet may represent different PSA-NCAM isoforms and/or PSA-NCAM with different post-translational modifications. When treated with neuraminidase to remove SA, the molecular weight of the PSA-NCAM triplet was reduced by an estimated 10-15 kDa (lane 2). FIG. 4B shows that PSA-NCAM was present as a triplet in two normal human sera (lane 1 and 2), and that this PSA-NCAM triplet was less distinguishable in the enriched HIBM sera (lane 3 and 4). In one HIBM patient (HIBM 2), the top bands of the PSA-NCAM triplet were substantially decreased in intensity and PSA-NCAM appeared as a single species of lower molecular weight (lane 4, arrow).

FIG. 4C shows that PSA-NCAM was detected by the 123C3 antibody as a triplet in two normal human sera (lanes 1 and 2). As shown in lanes 3-6, the lower band of the PSA-NCAM triplet appeared to migrate slightly faster in the sera of four different HIBM patients (arrow), indicating that it has a lower molecular weight.

FIG. 5 shows detection of PSA-NCAM in human serum using the C-20 antibody. A pattern of PSA-NCAM bands between 60 kDa and 150 kDa was observed in normal human (lanes 3 and 4). The major PSA-NCAM band had a MW of ˜130 kDa (arrow). Another major PSA-NCAM band of low molecular weight was detected at around 60 kDa. As shown in lanes 1 and 2, the intensity of both the 130 kDa and 60 kDa bands was significantly reduced in the sera of HIBM patients. Lane 5 shows normal serum treated with neuraminidase to remove SA; here, both the 130 kDa and 60 kDa bands were significantly reduced in intensity.

### DETAILED DESCRIPTION OF THE INVENTION

The present invention is based, in pertinent part, on the surprising discovery that alterations in sialylation states of polysialic acid-glycoproteins can be determined from a blood sample, and that these alterations represent a biomarker for conditions of sialic acid deficiency. Compared to previous technologies, which instead relied on muscle tissue biopsies, this discovery makes it less invasive and thus much easier to use the sialylation state of polysialic acid-glycoproteins as a biomarker for diagnosing conditions of sialic acid deficiency, determining whether a subject is suitable for sialic acid replacement therapy, regularly monitoring responsiveness or efficacy of a sialic acid replacement therapy in a subject, and using that information to improve treatment of such subjects, among other methods described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual (Sambrook et al., 3^{rd} Edition, 2000); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (D. Wild, ed., Stockton Press NY, 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellschaft mbH, 1993); and B. Perbal, A Practical Guide to Molecular Cloning (3^{rd} Edition 2010). All publications, patents and patent applications cited herein are hereby incorporated by reference in their entirety.

### DEFINITIONS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. Description referring to “about X” also includes description of “X.”

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, “a biological fluid sample” includes a blood, cerebrospinal fluid or urine sample which contains a molecule which is to be characterized and/or identified, for example, based on physical, biochemical, chemical physiological, and/or genetic characteristics. In certain embodiments, a biological fluid sample does not include a tissue biopsy sample, such as a muscle tissue biopsy sample. A “blood sample” includes a serum or plasma sample.

The terms “disorder” and “disease” are used interchangeably herein, and refer to any alteration in the state of the body or one of its organs and/or tissues, interrupting or disturbing the performance of organ function and/or tissue function (e.g., causes organ dysfunction) and/or causing a symptom such as discomfort, dysfunction, distress, or even death to a subject afflicted with the disease.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected.

In some embodiments, statistical significance is determined at a p-value of 0.1 or less, 0.05 or less, or 0.01 or less. In some embodiments, the p-value is between about any of 0.01 and 0.05 or 0.01 and 0.1. In specific cases, the significance level is defined at a p-value of 0.05 or less. In some embodiments, the p-values are corrected for multiple comparisons, for example, multiple comparisons can be corrected for using Bonferroni correction. In some embodiments, p-values are determined using permutation approaches, which are well known to those in the art. Permutation tests include randomization tests, re-randomization tests, exact tests, the jackknife, the bootstrap and other re-sampling schemes. In particular embodiments, the threshold criterion comprises a correlation value. In some embodiments, the correlation value is “r”, for instance, where “r” is greater than or equal to about any of 0.95, 0.90, 0.85, 0.80, 0.75, 0.70, 0.65, 0.60, 0.55, 0.50, 0.45, 0.40, 0.35, 0.30 or 0.25.

A “subject,” as used herein, includes any subject that has, is suspected of having, or is at risk for having a condition of sialic acid deficiency. Suitable subjects (or patients) include mammals, such as laboratory animals (e.g., mouse, rat, rabbit, guinea pig), farm animals, and domestic animals or pets (e.g., cat, dog). Non-human primates and, preferably, human patients, are included. A subject “at risk” may or may not have detectable disease, and may or may not have displayed detectable disease prior to the diagnostic or treatment methods described herein. “At risk” denotes that a subject has one or more so-called risk factors, which are measurable parameters that correlate with development of a condition of sialic acid deficiency, which are described herein. A subject having one or more of these risk factors has a higher probability of developing a sialic acid deficiency than a subject without these risk factor(s). One example of such a risk factor is reduced sialylation of one or more polysialic acid-glycoproteins, as determined from a tissue sample (e.g., muscle biopsy) or fluid sample (e.g., blood sample).

“Substantially” or “essentially” means nearly totally or completely, for instance, 95% or greater of some given quantity.

The term “therapeutically effective amount” as used herein, refers to the level or amount of one or more agents needed to treat a condition, or reduce or prevent injury or damage, optionally without causing significant negative or adverse side effects. For instance, a therapeutically effective amount includes an amount of a pharmaceutical formulation including one or more compounds in the sialic acid biosynthesis pathway sufficient to produce a desired therapeutic outcome (e.g., reduction of severity of a disease or condition).

A “prophylactically effective amount” refers to an amount of an agent (e.g., a pharmaceutical formulation including one or more compounds in the sialic acid biosynthesis pathway) sufficient to prevent or reduce severity of a future disease or condition when administered to a subject who is susceptible and/or who may develop a disease or condition.

The terms “treating” and “treatment” as used herein refer to an approach for obtaining beneficial or desired results including clinical results, and may include even minimal changes or improvements in one or more measurable markers of the disease or condition being treated. A treatment is usually effective to reduce at least one symptom of a condition, disease, disorder, injury or damage. Exemplary markers of clinical improvement will be apparent to persons skilled in the art. Examples include, but are not limited to, one or more of the following: decreasing the severity and/or frequency one or more symptoms resulting from the disease, diminishing the extent of the disease, stabilizing the disease (e.g., preventing or delaying the worsening of the disease), delay or slowing the progression of the disease, ameliorating the disease state, increasing production of sialic acid, the sialylation precursor CMP-sialic acid (e.g., increasing intracellular production of sialic acid) and restoring the level of sialylation in muscle and other proteins, decreasing the dose of one or more other medications required to treat the disease, and/or increasing the quality of life.

“Prophylaxis,” “prophylactic treatment,” or “preventive treatment” refers to preventing or reducing the occurrence or severity of one or more symptoms and/or their underlying cause, for example, prevention of a disease or condition in a subject susceptible to developing a disease or condition (e.g., at a higher risk, as a result of genetic predisposition, environmental factors, predisposing diseases or disorders, or the like). Prophylaxis includes prophylaxis of HIBM myopathy in which chronic disease changes in the muscles are irreversible, and for which animal model data suggests that prophylactic treatment prior to such irreversible damage confers a significant treatment benefit.

As used herein, “delaying the progression or development” of the disease means to defer, hinder, slow, retard, stabilize, and/or postpone development of the disease. This delay can be of varying lengths of time, depending on the history of the disease and/or individual being treated.

### Methods of Use

As noted above, certain embodiments provided herein include methods for diagnosing a condition of sialic acid deficiency in a subject comprising determining the sialylation state of a polysialic acid-glycoprotein in a blood sample from the subject, diagnosing the subject as sialic acid deficiency if the sialylation state is less than a pre-determined level. In certain of these and related embodiments, the methods further comprise recommending the subject for treatment of sialic acid deficiency.

Also included are methods of determining the level of sialic acid deficiency based on the level of decrease of the sialylation state from the pre-determined level. Typically, the greater the decrease of the sialylation state (i.e., hypo-sialylation) relative to the pre-determined level or other reference (e.g., level of a healthy subject, level of an earlier sample from the subject), the greater the risk or severity of the condition of sialic acid deficiency, and the more likely it is that a healthcare provide will recommend treatment, or optimize an existing treatment.

Certain embodiments include methods for monitoring responsiveness or efficacy of a treatment to a subject suffering from sialic acid deficiency comprising determining the sialylation state of a polysialic acid-glycoprotein in a blood sample from the subject, wherein an increase of the sialylation state (i.e., hyper-sialylation) of the polysialic acid-glycoprotein is indicative of responsiveness or efficacy of the treatment. Certain of these methods further comprise determining the future treatment regimen based on the sialylation state of the polysialic acid-glycoprotein in the blood sample. For instance, in some aspects, a determination of increased sialylation or hypo-sialylation relative to a reference or pre-determined level, such as an earlier sample from that same subject or a healthy control, could indicate that the current treatment is ineffective and optionally could be altered, for example, by increasing the dosage and/or frequency of the current therapeutic agent, adding another therapeutic agent, switching to a different therapeutic agent, or any combination thereof. In some aspects, a determination of no significant change in sialylation relative to an earlier sample from that same subject, such as an earlier hypo-sialylated sample (the latter being relative to a healthy control), could likewise indicate that the treatment is ineffective and optionally could be altered. In certain aspects, a determination of increased sialylation or hyper-sialylation relative to a reference or pre-determined level, such as an earlier sample from that same subject, could indicate that the current treatment is effective. Likewise, a determination of normal sialylation or hyper-sialylation relative to a healthy control could indicate that the current treatment is effective.

Some embodiments include methods for determining whether a subject is suitable for a sialic acid replacement therapy comprising determining the sialylation state of a polysialic acid-glycoprotein in a blood sample from the subject, wherein the subject is suitable for a sialic acid replacement therapy if the sialylation state of the polysialic acid-glycoprotein is less than a predetermined level and wherein the subject is not suitable for a sialic acid replacement therapy if the sialylation state of the polysialic acid-glycoprotein is equal to or higher than the pre-determined level. In these and related embodiments, the pre-determined level could be from a healthy subject or a population of healthy subjects (a healthy control), that is, a subject or population without a condition of sialic acid deficiency. In some instances, a determination of hypo-sialylation relative to a healthy control indicates that the individual is suitable for treatment with a therapeutic agent (e.g., sialic acid replacement therapy). In some embodiments, a determination of normal sialylation or hyper-sialylation relative to a healthy control indicates that the individual is not suitable for treatment with a therapeutic agent.

Also included are methods for treating a subject comprising determining the sialylation state of a polysialic acid-glycoprotein in a blood sample from the subject, and administering a sialic acid replacement therapy to the subject if the sialylation state of the polysialic acid-glycoprotein is less than a pre-determined level. Certain treatment methods may comprise (a) selecting an individual based upon a hypo-sialylation state of one or more polysialic acid-glycoproteins in a blood sample from the subject, relative to a reference or pre-determined level; and (b) administering to the selected subject an effective amount of a sialic acid replacement therapy. Exemplary therapies for treating conditions of sialic acid deficiency are described herein.

When sialylation is used as a basis for selection, assessing, measuring, or determining method of treatment and/or prevention as described herein, the marker is typically measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); or (f) predicting likelihood of clinical benefits. As would be well understood by one in the art, an evaluation of an individual's health-related quality of life in a clinical setting can be an indication that this parameter was used as a basis for initiating, continuing, and/or ceasing administration of the treatments described herein.

Some embodiments relate generally to methods of assaying the sialylation state of a polysialic acid-glycoprotein in a subject comprising obtaining or receiving a blood sample of the subject, and determining the sialylation state of a polysialic acid-glycoprotein in the blood sample. Exemplary assays include Western blot, ELISA and lectin chromatography, as described herein and known in the art. Often, the blood sample is from a subject that has, is at risk of having, or is suspected of having a condition of sialic acid deficiency, and the determination aids in diagnosing the subject, monitoring the treatment status of the subject, or determining whether the subject is suitable for a give sialic acid replacement therapy. In certain aspects, these assays are performed at a diagnostic laboratory, and the information is then provided to the subject or a physician or other healthcare provide. Particular embodiments thus include methods for providing data comprising determining the sialylation state of a polysialic acid-glycoprotein in a blood sample from a subject, and providing the information of the sialylation state to a healthcare provider for diagnosis or treatment of the subject.

Also included are a collection of molecular weight data comprising the molecular weight of a polysialic acid-glycoprotein from a blood sample of a testing subject. Certain of these embodiments may further comprise data on the molecular weight of the polysialic acid-glycoprotein of a control sample, such a different sample from the same subject (e.g., earlier blood sample, muscle tissue sample), or a blood or other sample from one or more control subjects (e.g., healthy subject or population of subjects). These data can indicate, for instance, the name of the subject, the date, the state of treatment of the subject, if available, the type sample (e.g., blood sample), and the molecular weight of one or more polysialic acid-glycoproteins, as described herein. These data can also indicate a preliminary result, for example, that one or more polysialic acid-glycoprotein(s) of interest are hypo-sialylated, have normal levels of sialylation, or are hyper-sialylated, relative to a reference or pre-determined value. These data can be in the form of a hard-copy or paper-copy, or an electronic form, such as a computer-readable medium.

As noted above, the methods provided herein are based on assaying for the sialylation state of a polysialic acid-glycoprotein. A polysialic acid-glycoprotein includes any combination of sialic acid and glycoprotein. It can also include any variety of proteins that are glycosylated with two or more sialic acid moieties at any one or more position(s) or amino acid residue(s) along the length of the protein, including, but not limited to, the N-terminus and/or the C-terminus of the polysialic acid-glycoprotein. Non-limiting examples include N-linked glycosylation, for instance, by attachment to a nitrogen of asparagine or arginine side-chains, and O-linked glycosylation, for instance, by attachment to the hydroxy oxygen of serine, threonine, tyrosine, hydroxylysine, or hydroxyproline side-chains. Certain consensus sequences for N-linked glycosylation include attached by an amide bond to an asparagine residue belonging to a consensus sequence NX(S/T), where X is any amino acid except proline.

A polysialic acid-glycoprotein can be a protein that is further characterized by one or more additional features, such as its tissue or cellular expression pattern or localization, solubility, or sialylation state, the latter being represented, for example, by the protein's molecular weight and/or sialylation pattern, as described herein.

In some embodiments, the polysialic acid-glycoprotein is expressed in muscle tissue, including a polysialic acid-glycoprotein that is released and/or secreted from a muscle cell (e.g., injured muscle cell). In some embodiments, the polysialic acid-glycoprotein is a transmembrane or GPI-anchored polysialic acid-glycoprotein which has been released from the cell membrane into a biological fluid. In some embodiments, the polysialic acid-glycoprotein is a secreted polysialic acid-glycoprotein.

In certain embodiments, the polysialic acid-glycoprotein is a soluble polysialic acid-glycoprotein. “Soluble” as used in this context means not associated with a cell (e.g., via a receptor) and/or not physically bound to a cell (e.g., not physically bound to a cell membrane) by a transmembrane domain or a lipid linker.

In certain embodiments, the polysialic acid-glycoprotein can be a protein that is characterized by its molecular weight. For instance, a polysialic acid-glycoprotein can have a molecular weight from about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250 or more kilodaltons (kDA), including all integers and ranges in between. Examples of ranges of molecular weights of a polysialic acid-glycoprotein include from about 50-70, 60-80, 70-90, 80-100, 90-110, 100-120, 110-130, 120-150, 130-160, 140-170, 150-180, 160-190, 170-200, 180-210, 190-210, 200-220, 210-230, 220-240, or 230-250 kDA. Certain polysialic acid-glycoproteins can have different isoforms or appear as multiple bands on a Western blot, with molecular weights ranging as described above, and/or ranging from between about 50-100, 50-110, 50-120, 50-130, 50-140, 50-150, 50-160, 50-170, 50-180, 50-190, 50-200, 60-100, 60-110, 60-120, 60-130, 60-140, 60-150, 60-160, 60-170, 60-180, 60-190, 60-200, 70-100, 70-110, 70-120, 70-130, 70-140, 70-150, 70-160, 70-170, 70-180, 70-190, 70-200, 80-100, 80-110, 80-120, 80-130, 80-140, 80-150, 80-160, 80-170, 80-180, 80-190, 80-200, 90-100, 90-110, 90-120, 90-130, 90-140, 90-150, 90-160, 90-170, 90-180, 90-190, 90-200, 100-110, 100-120, 100-130, 100-140, 100-150, 100-160, 100-170, 100-180, 100-190, or 100-200 kDA. Other ranges will be apparent to persons skilled in the art.

In some embodiments, a polysialic acid-glycoprotein can be a protein that is characterized by its sialylation pattern. Examples of sialylation patterns include the number of sialic acid modifications along the length of the protein (i.e., the number of amino acid residues having at least one sialic acid modification, e.g., N-linked, O-linked, C-terminus, N-terminus), for instance, as a single sialic acid residue attached to a given amino acid, a polysialic acid polymer attached to a given amino acid, or any combination thereof. In certain embodiments, a polysialic acid-glycoprotein may have about 1, 2, 3, 4, 5, 6, 7, 8, 9, 10, 11, 12, 13, 14, 15, 16, 17, 18, 19, 20, 21, 22, 23, 24, 25 or more sialic acid modifications along the length of the protein.

In these and related embodiments, a polysialic acid-glycoprotein may be a protein that comprises one or more polysialic acid polymers, or oligosaccharide chains. For example, a polysialic acid-glycoprotein may comprise about 1, 2, 3, 4, 5, 6, 7, 8, 9, 10, 11, 12, 13, 14, 15, 16, 17, 18, 19, 20, 21, 22, 23, 24, 25 or more polysialic acid polymers. In some embodiments, any given polysialic acid polymer may include from at least about 2, 3, 4, or 5 sialic acid residues to about 50 to 100 or more sialic acid residues. Particular examples are polysialic acid polymers (i.e., chains) that include from at least about 2, 3, 4, 5, 6, 7, 8, 9, 10, 11, 12, 13, 14, 15, 16, 17, 18, 19, 20, 21, 22, 23, 24, 25, 26, 28, 28, 29, 30, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 111, 112, 113, 114, 115, 116, 117, 118, 119, 120, 121, 122, 123, 124, 125, 126, 128, 128, 129, 130, 141, 142, 143, 144, 145, 146, 147, 148, 149, 150, 160, 170, 180, 190, 200, or more individual sialic acid residues, including all integers and ranges in between. Some examples include polysialic acid polymers that include about 2-6, 3-6, 4-6, 5-6, 2-8, 3-8, 4-8, 5-8, 6-8, 7-8, 2-10, 3-10, 4-10, 5-10, 6-10, 7-10, 8-10, 9-10, 2-12, 3-12, 4-12, 5-12, 6-12, 7-12, 8-12, 9-12, 10-12, 11-12, 2-14, 3-14, 4-14, 5-14, 6-14, 7-14, 8-14, 9-14, 10-14, 11-14, 12-14, 13-14, 2-16, 3-16, 4-16, 5-16, 6-16, 7-16, 8-16, 9-16, 10-16, 11-16, 12-16, 13-16, 14-16, 15-16, 2-18, 3-18, 4-18, 5-18, 6-18, 7-18, 8-18, 9-18, 10-18, 11-18, 12-18, 13-18, 14-18, 15-18, 16-18, 17-18, 2-20, 3-20, 4-20, 5-20, 6-20, 7-20, 8-20, 9-20, 10-20, 15-20, 10-30, 20-30, 10-40, 20-40, 30-40, 10-50, 20-50, 30-50, 40-50, 10-60, 20-60, 30-60, 40-60, 50-60, 10-70, 20-70, 30-70, 40-70, 50-70, 60-70, 10-80, 20-80, 30-80, 40-80, 50-80, 60-80, 70-80, 10-90, 20-90, 30-90, 40-90, 50-90, 60-90, 70-90, 80-90, 10-100, 20-100, 30-100, 40-100, 50-100, 60-100, 70-100, 80-100, 90-100, 10-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200 individual sialic acid residues.

Specific examples of polysialic acid-glycoproteins or other sialylated molecules include polysialic acid-neural cell adhesion molecule (PSA-NCAM; or NCAM), neprilysin, alpha-dystroglycan (alpha-DG), beta-dystroglycan (beta-DG), sarcoglycans, PC-1, biglycan, podocalyxin, gangliosides, sialylated ion channels, certain cytokines, and some immune globulins.

In particular embodiments, the polysialic acid-glycoprotein is PSA-NCAM (or NCAM). PSA-NCAM is a heterogeneous polysialic acid-glycoprotein of the Immunoglobulin(Ig) superfamily which mediates cell-cell and cell-substratum interactions. There are numerous alternatively-spliced PSA-NCAM mRNAs produced, giving a wide diversity of PSA-NCAM isoforms. See, e.g., Reyes et al., Mol Cell Biol. 11:1654-61 (1991). Transcription, translation and post-translational modifications of PSA-NCAM can be regulated in a developmental and cell-specific fashion. In mammalian brain, the glycosylphosphatidylinositol (GPI)-anchored 125-kDa isoform and transmembrane 140- and 160-kDa isoforms are primarily expressed. In muscle, three isoforms have been described: a GPI-anchored 125-kDa isoform and transmembrane 140- and 155-kDa isoforms. Walsh et al., Development 105:803-811 (1989). A secreted PSA-NCAM isoforms which includes an in-frame stop codon and thus prematurely terminates the coding sequence, generating a truncated N-CAM polypeptide has also been reported. Gower et al., Cell 55:955-964, 1988. Furthermore, intact transmembrane isoforms of PSA-NCAM can be released from the plasma membrane and present in cerebrospinal fluid. Olsen et al., Biochem J 295:833-840, 1993.

The polysialic acid-glycoprotein can therefore include any soluble PSA-NCAM isoform present in a blood sample of a subject. In some embodiments, the polysialic acid-glycoprotein includes a GPI-anchored or transmembrane isoform of PSA-NCAM which has been released from the plasma membrane into the blood of the subject. In some embodiments, the polysialic acid-glycoprotein includes a secreted PSA-NCAM. In some embodiments, the polysialic acid-glycoprotein includes a PSA-NCAM isoform that is about 60 kDA, 110 kDA, 120 kDA, 130 kDa, 140 kDA, or 150 kDA isoform of PSA-NCAM, or one or more isoforms in the 60-150 kDA, 100-150 kDA, or 110-130 kDA range, including any other range described herein, or any combination thereof. In specific embodiments, the polysialic acid-glycoprotein includes an triplet repeat pattern of PSA-NCAM that ranges, for example, from about 110-130 kDA. In some aspects, hyposialylation can be detected by alterations in this PSA-NCAM triplet repeat pattern, such as reductions in one or more of the individual bands, or altered migration patterns of one or more of the bands (e.g., the lower band of the N-CAM triplet can migrate faster (indicative of lower MW) in certain conditions of sialic acid deficiency).

In some embodiments, the polysialic acid-glycoprotein includes an alpha-dystroglycan (α-DG). α-DG is an essential component of the dystrophin-glycoprotein complex. Michele et al. Nature 418, 417-422 (2002); Michele et al. J Biol Chem 278, 15457-15460 (2003). α-DG is heavily glycosylated with O-mannosyl glycans (mannose-N-acetylglucosamine-galactose-sialic acid) linked to a serine or threonine; these glycans are critical for α-DG's interactions with laminin and other extracellular ligands. In some embodiments, the polysialic acid-glycoprotein includes a soluble α-DG. In some embodiments, the polysialic acid-glycoprotein includes an α-DG released from the plasma membrane into a biological fluid, such as the blood.

In certain embodiments, the polysialic acid-glycoprotein is neprilysin. Neprilysin is a zinc-dependent metalloprotease enzyme that degrades a number of small secreted peptides, most notably the amyloid beta peptide. Synthesized as a membrane-bound protein, the neprilysin ectodomain is released into the extracellular domain after it has been transported from the Golgi apparatus to the cell surface. In some embodiments, the polysialic acid-glycoprotein is a soluble neprilysin. In some embodiments, the polysialic acid-glycoprotein is neprilysin released from the plasma membrane into a biological fluid, such as the blood.

The sialylation state of a polysialic acid-glycoprotein can be determined based on a variety of parameters. In certain embodiments, the sialylation state of a polysialic acid-glycoprotein is determined based on its molecular weight, as described herein. In some embodiments, the sialylation state of the polysialic acid-glycoprotein is determined based on the sialylation pattern, as described herein, including the number or degree or type of sialic acid modifications. The sialylation state can also be determined based on the presence or absence or levels of a specific sialic acid modification, identified, for example, by an antibody that specifically binds to one or more sites of sialic acid modification (e.g., the antibody binds to the un-sialylated site but not the sialylated site, or vice versa).

A polysialic acid-glycoprotein of the subject can be hyper-sialylated, hypo-sialylated, or have no detectable or significant alteration in sialylation, relative to one or more reference or pre-determined levels. Hence, in certain embodiments, the determination of a sialylation state as “hyper-” or “hypo-” may depend on a given particular context, for instance, the relationship of the determined sialylation state to a reference or pre-determined level; and the information provided by that determination may likewise depend on the context.

Accordingly, for the diagnostic, treatment monitoring, and related methods, the sialylation state of a polysialic acid-glycoprotein is typically determined relative to a reference or pre-determined level or value. In some embodiments, the reference or pre-determined level is based on the sialylation state (e.g., number or degree of sialic acid modifications, molecular weight) of one or more polysialic acid-glycoproteins from a healthy subject or a population of healthy subjects, that is, a subject or population of subjects without a condition of sialic acid deficiency. In other embodiments, the reference or pre-determined level is based on the sialylation state (e.g., number or degree of sialic acid modifications, molecular weight) of one or more polysialic acid-glycoproteins from a subject or population of subjects having a condition of sialic acid deficiency, which optionally does not include the subject being tested.

In particular embodiments, the reference or pre-determined level is based on the sialylation state (e.g., number or degree of sialic acid modifications, molecular weight) of one or more polysialic acid-glycoproteins from a different sample from the subject being tested. Examples include a sample taken from the subject at an earlier or later time-point, and samples taken from other tissue (e.g., muscle), optionally where the subject has, is at risk for having, or is suspected of having a condition of sialic acid deficiency. In certain embodiments, the subject being tested has a condition of sialic acid deficiency and is currently undergoing sialic acid replacement therapy; the sample can be taken from the subject before, during, or after such treatment.

In some embodiments, the reference or pre-determined level is the median level of sialylation of one or more polysialic acid-glycoproteins from the subject or population of subjects, as described herein. In some embodiments, the reference or pre-determined level is the fraction of the total unsialylated or lowly sialylated polysialic acid-glycoproteins relative to the fraction of the highly sialylated polysialic acid-glycoproteins in the subject or population of subjects. In particular embodiments, the reference or pre-determined level is based on the sialylation pattern of the polysialic acid-glycoprotein(s), as described herein, including the number or degree of sialic acid modifications. In specific embodiments, the reference or pre-determined level is based on the molecular weight of the polysialic acid-glycoprotein(s), as described herein; i.e., the reference is a pre-determined molecular weight. In some embodiments, the information for the reference or pre-determined level is derived from a blood sample of the subject or population of subjects described herein. In specific embodiments, the reference or pre-determined level is based on the sialylation state (e.g., number or degree of sialic acid modifications, molecular weight) of transferrin or PSA-NCAM.

A “hyper-sialylated” protein has an “increased” sialylation state relative to a reference or pre-determined level, which can include, for example, an increase of about 1.25×, 1.5×, 1.75×, 2×, 3×, 4×, 5×, 6×, 7×, 8×, 9×, 10×, 20×, 30×, 40×, 50×, 60×, 70×, 80×, 90×, 100×, 200×, 300×, 400×, 500×, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%, 200%, 300%, 400%, 500%, 600%, 700%, 800%, 900%, 1000% or more relative to the reference or pre-determined level. In certain embodiments, the increase relative to the reference or pre-determined level is statistically significant.

In some embodiments, a hyper-sialylated protein can have an increased molecular weight relative to a reference or pre-determined value. For example, a hyper-sialylated protein can have an increase in molecular weight of about 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100 or more kDA relative to the reference or pre-determined value, including all integers and ranges in between. In some embodiments, a hyper-sialylated protein can have an increase in molecular weight ranging from about 5-10, 6-10, 7-10, 8-10, 9-10, 2-12, 3-12, 4-12, 5-12, 6-12, 7-12, 8-12, 9-12, 10-12, 11-12, 2-14, 3-14, 4-14, 5-14, 6-14, 7-14, 8-14, 9-14, 10-14, 11-14, 12-14, 13-14, 5-15, 10-15, 2-16, 3-16, 4-16, 5-16, 6-16, 7-16, 8-16, 9-16, 10-16, 11-16, 12-16, 13-16, 14-16, 15-16, 2-18, 3-18, 4-18, 5-18, 6-18, 7-18, 8-18, 9-18, 10-18, 11-18, 12-18, 13-18, 14-18, 15-18, 16-18, 17-18, 2-20, 3-20, 4-20, 5-20, 6-20, 7-20, 8-20, 9-20, 10-20, 15-20, 10-30, 20-30, 10-40, 20-40, 30-40, 10-50, 20-50, 30-50, 40-50, 10-60, 20-60, 30-60, 40-60, 50-60, 10-70, 20-70, 30-70, 40-70, 50-70, 60-70, 10-80, 20-80, 30-80, 40-80, 50-80, 60-80, 70-80, 10-90, 20-90, 30-90, 40-90, 50-90, 60-90, 70-90, 80-90, 10-100, 20-100, 30-100, 40-100, or 50-100 kDA, relative to the reference or predetermined level.

A hyper-sialylated protein can have an increased number of sialic acid modifications relative to a reference or pre-determined level. For example, such a protein can have an increase of about 1, 2, 3, 4, 5, 6, 7, 8, 9, 10, 11, 12, 13, 14, 15, 16, 17, 18, 19, 20, 21, 22, 23, 24, 25 or more sialic acid modifications (e.g., single sialic acid residues, polysialic acid polymers/chains) along the length of the protein, relative to the reference or pre-determined level. In certain embodiments, a hyper-sialylated protein can have an increase of about 1, 2, 3, 4, 5, 6, 7, 8, 9, 10, 11, 12, 13, 14, 15, 16, 17, 18, 19, 20, 21, 22, 23, 24, 25 or more polysialic acid polymers, where the polymer(s) range in size from about 2, 3, 4, 5, 6, 7, 8, 9, 10, 11, 12, 13, 14, 15, 16, 17, 18, 19, 20, 21, 22, 23, 24, 25, 26, 28, 28, 29, 30, 41, 42, 43, 44, 45, 46, 47, 48, 49, 50, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more sialic acid residues per polymer, relative to the reference or pre-determined level. In certain embodiments, a hyper-sialylated protein may have an increase in the number of sialic acid residues contained in one or polysialic acid polymers or oligosaccharide chains, including an increase of about 2, 3, 4, 5, 6, 7, 8, 9, 10, 11, 12, 13, 14, 15, 16, 17, 18, 19, 20, 21, 22, 23, 24, 25, 26, 28, 28, 29, 30, 41, 42, 43, 44, 45, 46, 47, 48, 49, 50 sialic acid residues in a given polysialic acid polymer, relative to the reference or pre-determined level.

A “hypo-sialylated” protein has a “decreased” sialylation state relative to a reference or pre-determined level, which can include, for example, a decrease of about 1.25×, 1.5×, 1.75×, 2×, 3×, 4×, 5×, 6×, 7×, 8×, 9×, 10×, 20×, 30×, 40×, 50×, 60×, 70×, 80×, 90×, 100×, 200×, 300×, 400×, 500×, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% or more relative to the reference or pre-determined level. In certain embodiments, the decrease relative to the reference or pre-determined level is statistically significant.

In some embodiments, a hypo-sialylated protein can have a decreased molecular weight relative to a reference or pre-determined value. For example, a hypo-sialylated protein can have decrease in molecular weight of about 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100 or more kDA relative to reference or pre-determined value, including all integers and ranges in between. In some embodiments, a hypo-sialylated protein can have a decrease in molecular weight ranging from about 5-10, 6-10, 7-10, 8-10, 9-10, 2-12, 3-12, 4-12, 5-12, 6-12, 7-12, 8-12, 9-12, 10-12, 11-12, 2-14, 3-14, 4-14, 5-14, 6-14, 7-14, 8-14, 9-14, 10-14, 11-14, 12-14, 13-14, 5-15, 10-15, 2-16, 3-16, 4-16, 5-16, 6-16, 7-16, 8-16, 9-16, 10-16, 11-16, 12-16, 13-16, 14-16, 15-16, 2-18, 3-18, 4-18, 5-18, 6-18, 7-18, 8-18, 9-18, 10-18, 11-18, 12-18, 13-18, 14-18, 15-18, 16-18, 17-18, 2-20, 3-20, 4-20, 5-20, 6-20, 7-20, 8-20, 9-20, 10-20, 15-20, 10-30, 20-30, 10-40, 20-40, 30-40, 10-50, 20-50, 30-50, 40-50, 10-60, 20-60, 30-60, 40-60, 50-60, 10-70, 20-70, 30-70, 40-70, 50-70, 60-70, 10-80, 20-80, 30-80, 40-80, 50-80, 60-80, 70-80, 10-90, 20-90, 30-90, 40-90, 50-90, 60-90, 70-90, 80-90, 10-100, 20-100, 30-100, 40-100, or 50-100 kDA, relative to the reference or predetermined level.

A hypo-sialylated protein can have a decreased number of sialic acid modifications relative to a reference or pre-determined level. For example, such a protein can have a decrease of about 1, 2, 3, 4, 5, 6, 7, 8, 9, 10, 11, 12, 13, 14, 15, 16, 17, 18, 19, 20, 21, 22, 23, 24, 25 or more sialic acid modifications (e.g., single sialic acid residues, polysialic acid polymers) along the length of the protein, relative to the reference or pre-determined level. In certain embodiments, a hypo-sialylated protein can have a decrease of about 1, 2, 3, 4, 5, 6, 7, 8, 9, 10, 11, 12, 13, 14, 15, 16, 17, 18, 19, 20, 21, 22, 23, 24, 25 or more polysialic acid polymers, where the polymer(s) range in size from about 2, 3, 4, 5, 6, 7, 8, 9, 10, 11, 12, 13, 14, 15, 16, 17, 18, 19, 20, 21, 22, 23, 24, 25, 26, 28, 28, 29, 30, 41, 42, 43, 44, 45, 46, 47, 48, 49, 50, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more sialic acid residues per polymer, relative to the reference or pre-determined level. In certain embodiments, a hypo-sialylated protein may have a decrease in the number of sialic acid residues contained in one or polysialic acid polymers, including decrease of about 2, 3, 4, 5, 6, 7, 8, 9, 10, 11, 12, 13, 14, 15, 16, 17, 18, 19, 20, 21, 22, 23, 24, 25, 26, 28, 28, 29, 30, 41, 42, 43, 44, 45, 46, 47, 48, 49, 50 sialic acid residues in a given polysialic acid polymer, relative to the reference or pre-determined level.

In some embodiments, the measured levels of one or more sialylated polysialic acid-glycoproteins in a biological fluid sample are normalized. For example, the levels are normalized against a control (e.g., a control protein) in the biological fluid sample, the level of which does not change (or does not change significantly) among different samples. In some embodiments, the levels are normalized against a control blood protein, the level of which does not change (or does not change significantly) among different samples. In some embodiments, the control blood protein is albumin, transferrin, or immunoglobulin.

The sialylation state of a polysialic acid-glycoprotein can be determined or assayed according to routine techniques in the art. As one example, the sialylation state of a polysialic acid-glycoprotein can be determined using one or more antibodies. In these and related embodiments, the sialylation state of a polysialic acid-glycoprotein may be determined by SDS-PAGE and Western blot using an antibody to the polysialic acid-glycoprotein, which is optionally capable of detecting both sialylated and unsialylated polysialic acid-glycoprotein. In some embodiments, the sialylation state of a polysialic acid-glycoprotein may be determined by an enzyme-linked immunosorbent assays (ELISA), such as a sandwich ELISA. One example of an ELISA may use a first antibody that binds to a non-sialylated region of the polysialic acid-glycoprotein and a second anti-sialic acid antibody, thereby detecting the presence and level of sialylated polysialic acid-glycoprotein. Another example of an ELISA uses a first antibody that binds to a sialylated region of the polysialic acid-glycoprotein and a second antibody that binds to the non-sialylated region of the polysialic acid-glycoprotein. Certain embodiments may employ lectin affinity chromatography (see, e.g., Freeze, Curr Protoc Protein Sci. Chapter 9:Unit 9.1, 2001), using certain lectins that preferentially bind to sialic acid and which can thus be used to distinguish the sialylation state(s) of one or more polysialic acid-glycoproteins. In some embodiments, the sialylation state is determined by mass spectrometry methods such as MALDI-TOF (e.g., by methods described in WO 2007/124750 incorporated herein in its entirety). In some embodiments, the sialylation state is not determined by mass spectrometry methods such as MALDI-TOF. Other methods to determine the sialylation state of a polysialic acid-glycoprotein include, but are not limited to, size exclusion or affinity resin chromatography methods, affinity beads, filtration/isolation columns or centrifugation methods known in the art to separate fractions and isolate and/or purify proteins.

As noted above, a blood sample includes, for example, a directly isolated blood sample, a plasma sample, and a serum sample. Blood samples can be obtained according to routine techniques in the art, for example, venous blood draw or finger prick, and then analyzed immediately, or frozen or stored under refrigeration or on ice prior to analysis. Blood samples can be directly assayed for determining the sialylation state of a polysialic acid-glycoprotein, or they can be first concentrated or enriched prior to such determination. For instance, blood samples can be treated with one or more reagents or purification and/or extraction systems to separate, enrich, and/or isolate polysialic acid-glycoproteins including soluble polysialic acid-glycoproteins (e.g., using Mem-PER®). In some embodiments, a blood sample can be enriched to increase the ratio of polysialic acid-glycoproteins to other components in the sample. As another example, a blood sample can be enriched to increase the ratio of polysialic acid-glycoproteins to other components (e.g., other, non-polysialic acid-containing glycoproteins) in the sample. In certain embodiments, a blood sample can be treated to reduce certain components, for example, albumin and/or IgG, which might otherwise effect the analysis.

In certain embodiments, the blood sample is prepared using Mem-PER® Eukaryotic Membrane Protein Extraction Reagent Kit. In certain instances, the one or more reagents or purification and/or extraction systems separate, enrich and/or isolate glycoproteins including soluble polysialic acid-glycoprotein based on hydrophobicity. In some embodiments, the polysialic acid-glycoprotein including soluble glycoprotein is detected in the hydrophobic fraction. In some embodiments, the polysialic acid-glycoprotein including soluble glycoprotein is detected in the hydrophilic fraction.

A condition of sialic acid deficiency includes any disease, disorder, or condition associated with reduced sialylation state of a given polysialic acid-glycoprotein. Certain embodiments include myopathies that are associated with sialic acid deficiency. A myopathy is a muscular disease in which the muscle fibers do not function for any one of many reasons, typically resulting in muscular weakness. Examples of myopathies associated with sialic acid deficiency include Hereditary Inclusion Body Myopathy (HIBM), Nonaka myopathy, and Distal Myopathy with Rimmed Vacuoles (DMRV).

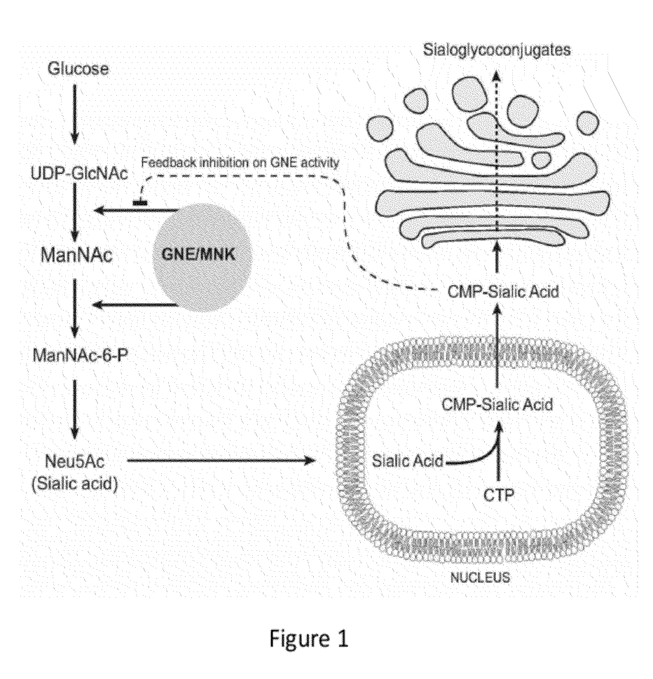

As noted above, certain embodiments include treatment of a condition of sialic acid deficiency, and related therapeutic agents and pharmaceutical compositions/formulations. Non-limiting examples of such treatments include replacement therapies, which typically achieve increased sialic acid levels by administering an agent that directly or indirectly increases one or more components of the sialic acid biosynthesis pathway (see, e.g., FIG. 1). Also included are gene therapies that incorporate one or more genes involved directly or indirectly in the sialic acid biosynthesis pathway.

Exemplary components of the sialic acid biosynthesis pathway include mannosamine, N-acetyl mannosamine (ManNAc), ManNac-6-phosphate (ManNAc-6-P), UDP-GlcNAc, N-acetylneuraminic acid (NeuAc), NeuAc-9-phosphate (NeuAc-9-P), sialic acid (i.e., 5-N-acetylneuraminic acid), and CMP-sialic acid. Hence, certain treatments include the direct administration of one or more of these components as compounds, or as derivatives or pharmaceutically acceptable salts thereof, including extended release formulations of such compounds (see, e.g., U.S. Application No. 61/363,995; and PCT/US2011/043910, each of which is incorporated by reference in its entirety). The term “derivative” as used herein includes derivatives, analogs, prodrugs, and unnatural precursors of a given compound. In specific embodiments, the compound in the sialic acid biosynthesis pathway or a derivative thereof does not include glucose or a pharmaceutically acceptable salt thereof.

As one example, the one or more compounds in the sialic acid biosynthesis pathway or derivative thereof include ManNAc or a derivative thereof (see, e.g., U.S. Application No. 2010/0249047 and WO 200/8150477, which are incorporated by reference in their entireties). Structures of such ManNAc and derivatives thereof include, but are not limited to, those defined by the formula below:

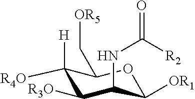

wherein: R₁, R₃, R₄, or R₅ is hydrogen, lower alkanoyl, carboxylate or lower alkyl; and R₂ is lower alkyl, lower alkanoylalkyl, lower alkyl alkanoyloxy.

The term lower alkyl refers to (C₁-C₆)alkyl. A lower alkyl includes methyl, ethyl, propyl, isopropyl, butyl, iso-butyl, sec-butyl, pentyl, 3-pentyl, hexyl, (C₃-C₆)cycloalkyl (e.g., cyclopropyl, cyclobutyl, cyclopentyl, or cyclohexyl), (C₃-C₆)cycloalkyl(C₁-C₆)alkyl (e.g., cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl), (C₁-C₆)alkoxy (e.g., methoxy, ethoxy, propoxy, isopropoxy, butoxy, iso-butoxy, sec-butoxy, pentoxy, 3-pentoxy, or hexyloxy) (C₂-C₆)alkenyl (e.g., vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl), (C₂-C₆)alkynyl (e.g., ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl), (C₁-C₆)alkanoyl (e.g., acetyl, propanoyl or butanoyl), halo(C₁-C₆)alkyl (e.g., iodomethyl, bromomethyl, chloromethyl, fluoromethyl, trifluoromethyl, 2-chloroethyl, 2-fluoroethyl, 2,2,2-trifluoroethyl, or pentafluoroethyl), hydroxy(C₁-C₆)alkyl (e.g., hydroxymethyl, 1-hydroxyethyl, 2-hydroxyethyl, 1-hydroxypropyl, 2-hydroxypropyl, 3-hydroxypropyl, 1-hydroxy butyl, 4-hydroxybutyl, 1-hydroxypentyl, 5-hydroxypentyl, 1-hydroxyhexyl, or 6-hydroxyhexyl), (C₁-C₆)alkoxycarbonyl (e.g., methoxycarbonyl, ethoxycarbonyl, propoxycarbonyl, isopropoxycarbonyl, butoxycarbonyl, pentoxycarbonyl, or hexyloxycarbonyl), (C₁-C₆)alkylthio (e.g., methylthio, ethylthio, propylthio, isopropylthio, butylthio, isobutylthio, pentylthio, or hexylthio), and/or (C₂-C₆)alkanoyloxy (e.g., acetoxy, propanoyloxy, butanoyloxy, isobutanoyloxy, pentanoyloxy, or hexanoyloxy).

In some embodiments, R₂ is methyl, and R₁, R₃, R₄, and R₅ is hydrogen. In some embodiments, the ManNAc or derivative thereof is N-acetyl mannosamine (ManNAc). In some embodiments, the ManNAc or derivative thereof is N-levulinoylmannosamine (ManLev) or N-azidoacetylmannosamine (ManNAz).

In some embodiments, the one or more compounds in the sialic acid biosynthesis pathway or derivative thereof include N-acetylneuraminic acid (NeuAc) or a derivative thereof. Structures of such NeuAc or derivatives thereof include, but are not limited to, those defined by the formula below:

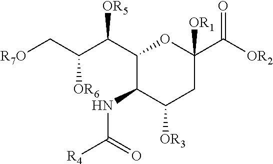

wherein each R₁, R₂, R₃, R₅, R₆, or R₇ is independently hydrogen, lower alkanoyl, carboxylate or lower alkyl; and R₄ is lower alkyl, lower alkanoylalkyl or lower alkyl alkanoyloxy.

In some embodiments, the one or more compounds in the sialic acid biosynthesis pathway or derivative thereof include sialic acid or a derivative thereof. In some embodiments, the sialic acid or derivative thereof is sialic acid. In some embodiments, the sialic acid or derivative thereof is a sialic acid analog such as N-levulinoyl sialic acid (SiaLev) or N-azidoacetyl sialic acid (SiaNAz). In some embodiments, the sialic acid or derivative thereof is bound as a glycoconjugate. In some embodiments, the sialic acid or derivative thereof is an unnatural precursor such as sialylactose. In some embodiments the sialic acid or derivative thereof is conjugated to an immunoglobulin. Specific embodiments include a sialic acid extended release formulation (see, e.g., U.S. Application No. 61/363,995; and PCT/US2011/043910). In specific embodiments, the extended release formulation is a formulation of Table 1, below.

TABLE 8
Quantitative Formula for Sialic Acid 325 mg and 500 mg sustained release
Tablets Prototypes.

| | | mg/Tablet | mg/Tablet | | | g/batch |
|---|---|---|---|---|---|---|
| | | ProCR | ProCR | | g/batch | 1800 g |
| Ingredient | Vendor | Hypromellose | Polyox | % w/w | 50 g size | size |
| Sialic Acid (N- | Food and | 325.0 | 325.0 | 43.3 | 21.65 | 779.4 |
| Acetylneuraminic | BioResearch | | | | | |
| acid) | Center, Inc | | | | | |
| Hypromellose, | Colorcon | 191.3 | — | 25.5 | 12.75 | 459 |
| Type 2208 | | | | | | |
| (Methocel ® K100 | | | | | | |
| M Premium CR) | | | | | | |
| Polyethylene Oxide | | — | 191.3 | 25.5 | 12.75 | 459 |
| WSR (Polyox) | | | | | | |
| Sodium Alginate | FMC | 159.0 | 159.0 | 21.2 | 10.60 | 381.6 |
| (Protanal ® LF | Biopolymer | | | | | |
| 120M) | | | | | | |
| Carrageenan | FMC | 31.5 | 31.5 | 4.2 | 2.10 | 75.6 |
| (Viscarin GP-209) | Biopolymer | | | | | |
| Microcrystallline | JRS Pharma | 39.8 | 39.8 | 5.3 | 2.65 | 95.4 |
| Cellulose and | | | | | | |
| Colloidal Sillicon | | | | | | |
| Dioxide (ProSolv ® | | | | | | |
| SMCC HD 90) | | | | | | |
| Magnesium | Mallinckrodt | 3.8 | 3.8 | 0.5 | 0.25 | 9.0 |
| Stearate | | | | | | |
| (HyQual ®), | | | | | | |
| Vegatable Source | | | | | | |
| Product Code 2257 | | | | | | |

| | | | | | |
|---|---|---|---|---|---|
| Total for 325 mg Strength | 750.4 | 750.4 | 100 | 50 | 1800 |
| Total for 500 mg Strength | 1154.5 | 1154.8 | 100 | — | — |

In one variation, the one or more compounds in the sialic acid biosynthesis pathway or derivative thereof is an ester of a compound in the sialic acid biosynthesis pathway. In one aspect, the one or more compounds in the sialic acid biosynthesis pathway or derivative thereof is an ester of sialic acid or MaNAc. In a particular variation, the one or more compounds in the sialic acid biosynthesis pathway or derivative thereof is an ester of sialic acid. In one aspect, the one or more compounds in the sialic acid biosynthesis pathway or derivative thereof is a prodrug of sialic acid. See also WO 2010/131712, published Nov. 18, 2010, for derivatives of compounds in the sialic acid biosynthesis pathway, which is incorporated herein by reference in its entirety and specifically with respect to compounds (e.g., derivatives of compounds in the sialic acid biosynthesis pathway) detailed therein.

In one aspect, a derivative of one or more compounds in the sialic acid biosynthesis pathway (e.g., a derivative of sialic acid or MaNAc) is an effective substrate replacement for sialic acid, such as in an subject who has or is suspected of having a condition of sialic acid deficiency. A derivative of one or more compounds in the sialic acid biosynthesis pathway (e.g., a derivative of sialic acid or MaNAc), or an extended release formulation comprising a derivative of one or more compounds in the sialic acid biosynthesis pathway (e.g., a derivative of sialic acid or MaNAc) may exhibit any one or more of the following characteristics: (i) capable of delivering to an individual in need thereof a therapeutically effective amount of one or more compounds in the sialic acid pathway or derivatives thereof over a period of greater than about any of 1, 2, 3, 4, 5, 6, 7, 8, 9, 10, 11, 12, 13, 14, 15, 16, 17, 18, 19, 20, 21, 22, 23, or 24 hours; (ii) capable of delivering to an individual in need thereof a substantially constant therapeutically effective amount of one or more compounds in the sialic acid pathway or derivatives thereof over a period of greater than about any of 1, 2, 3, 4, 5, 6, 7, 8, 9, 10, 11, 12, 13, 14, 15, 16, 17, 18, 19, 20, 21, 22, 23, or 24 hours; (iii) capable of delivering to an individual in need thereof a therapeutically effective amount of one or more compounds in the sialic acid pathway or derivatives thereof with a Tₘₐₓ of between about any of 2-4 hours, 3-4 hours, 6-8 hours, 6-12 hours, 6-15 hours, 12-18 hours, or 18-24 hours; (iv) capable of delivering to an individual in need thereof a therapeutically effective amount of one or more compounds in the sialic acid pathway or derivatives thereof with a Cₘₐₓ of about 0.5-100 μg/mL; (v) capable of delivering to an individual in need thereof a therapeutically effective amount of one or more compounds in the sialic acid pathway or derivatives thereof with a trough level of about 0.1-20 μg/mL; (vi) capable of delivering to an individual in need thereof a therapeutically effective amount of one or more compounds in the sialic acid pathway or derivatives thereof with less than about any of 10%, 20%, 30%, 40%, 50%, 60%, or 70% excreted after one hour; (vii) capable of delivering to an individual in need thereof between about any of 0.01-750 mg/kg/day, 0.5-500 mg/kg/day, 1-250 mg/kg/day, 2.5-100 mg/kg/day, or 5-50 mg/kg/day of one or more compounds in the sialic acid pathway or derivatives thereof or a pharmaceutically acceptable salt of the foregoing; (viii) capable of delivering to an individual in need thereof between about any of 0.01-750 mg/kg/day, 0.5-500 mg/kg/day, 1-250 mg/kg/day, 2.5-100 mg/kg/day, or 5-50 mg/kg/day of one or more compounds in the sialic acid pathway or derivatives thereof or a pharmaceutically acceptable salt of the foregoing; (ix) has an absolute bioavailability of about 1 to about 50%; (x) has a bioavailability based on sialic acid levels in the urine of about 0.5 to about 100%; and (xi) has a mean residence time (MRT) of at least about 3.5 hours.

As noted above, gene replacement therapy is also contemplated. Any gene involved (e.g., directly, indirectly) in the sialic acid biosynthesis pathway can be utilized (see FIG. 1). As one example, certain embodiments include methods for increasing sialic acid production by providing a subject with a wild-type GNE-encoding nucleic acid sequence that is optionally operably linked to a regulatory element, such as a promoter and/or enhancer sequence (see U.S. Application No. 2011/027373; WO 2008/097623; and U.S. Application No. 2009/029811, which are incorporated by reference in their entireties). This gene replacement therapy targets GNE/MNK, which is defective in HIBM patients, typically due to an autosomal recessive mutation of the GNE gene (see, e.g., Nemunaitis et al., The Journal of Gene Medicine 12:403-12, 2010). The GNE gene encodes the bi-functional enzyme UDP-GlcNAc 2-epimerase/ManNAc kinase.

Various viral vectors that can be utilized for gene replacement therapy include adenovirus, herpes virus, vaccinia, adeno-associated virus (AAV), or, preferably, an RNA virus such as a retrovirus. Preferably, the retroviral vector is a derivative of a murine or avian retrovirus, or is a lentiviral vector. The preferred retroviral vector is a lentiviral vector. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of additional retroviral vectors can incorporate multiple genes. All of these vectors can transfer or incorporate a gene for a selectable marker so that transduced cells can be identified and generated.

“Non-viral” delivery techniques for gene therapy can also be used including, for example, DNA-ligand complexes, adenovirus-ligand-DNA complexes, direct injection of DNA, CaPO₄ precipitation, gene gun techniques, electroporation, liposomes, lipofection, and the like. Any of these methods are widely available to one skilled in the art and would be suitable for use in the present invention. Other suitable methods are available to one skilled in the art, and it is to be understood that the present invention can be accomplished using any of the available methods of transfection. Lipofection can be accomplished by encapsulating an isolated DNA molecule within a liposomal particle and contacting the liposomal particle with the cell membrane of the target cell. Liposomes are self-assembling, colloidal particles in which a lipid bilayer, composed of amphiphilic molecules such as phosphatidyl serine or phosphatidyl choline, encapsulates a portion of the surrounding media such that the lipid bilayer surrounds a hydrophilic interior. Unilammellar or multilammellar liposomes can be constructed such that the interior contains a desired DNA molecule.

A desirable clinical or non-clinical outcome of the treatment(s) described herein includes, but is not limited to, increased production of sialic acid, restored level of sialylation in muscle and other proteins, increased muscle function, increased muscle strength (e.g., muscle strength of the quadriceps), increased muscle tensile force, improved muscle movement, improved limb movement, muscle growth, increased muscle stamina, decrease in muscle fatigability, decrease in muscle atrophy, decrease in neuronal atrophy, increase in pulmonary function, reduction in proteinuria (e.g., lower amounts of protein in the urine), reduction in hematuria (e.g., lower amounts of red blood cells in the urine) increased activity, stable disease (e.g., preventing or delaying the worsening of the disease), and/or increase or elongation of overall survival. The clinical outcome(s) will then be considered, and a decision as to whether the patient is suitable for the therapy will be made accordingly, taking into account the patient's specific situation and the relevance of the clinical or non-clinical outcomes.

### Formulations and Administration

Various pharmaceutical formulations comprising one or more therapeutic agents may be used in any of the methods described herein. In particular, provided herein are pharmaceutical formulations comprising one or more therapeutic agents (e.g., those described herein) and a pharmaceutically acceptable carrier, diluent, and/or excipient. Examples of suitable carriers, excipients, and diluents include, but are not limited to, sugars, lactose, dextrose, sucrose, sorbitol, mannitol, starches, gum such as xanthan gum, guar gum, or gum acacia, calcium phosphate, alginates, tragacanth, gelatin, calcium silicate, microcrystalline cellulose, polyethylene glycols, polyvinylpyrrolidone, phospholipics, cellulose, water, saline solution, syrup, methylcellulose, methyl- and propylhydroxybenzoates, talc, magnesium stearate, mineral oil, lubricating agents, wetting agents, emulsifying and suspending agents, preserving agents, sweetening agents, disintegrating agents, antioxidants, surfactants, and/or flavoring agents.

Pharmaceutical formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The pharmaceutical formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use.

In some embodiments, when the therapeutic agent is a nucleic acid, the therapeutic agent may be used and delivered to a system in connection with an appropriate delivery vehicle (such as a liposome or lipid nanoparticle). In specific aspects, the nucleic acid is administered in conjunction with a lipid nanoparticle. Particular embodiments include a human non-viral GNE-plasmid embedded in cationic liposomes (e.g., GNE Lipoplex). Merely for illustrative purposes, these and other embodiments can be administered via intramuscular injection (e.g., biceps and extensor carpi radialis longus), intravenously (IV), or via intrahepatic (the major organ of SA synthesis) injections.

For topical administration, the pharmaceutical formulation may be a cream, milk, gel, dispersion, or microemulsions, lotion thickened to a greater or lesser extent, impregnated pad, ointment or stick, aerosol formulations (e.g., sprays or foams), soaps, detergents, lotions or cakes of soap.

The pharmaceutical formulation may be a food supplement or incorporated into food or drink item such as a nutritional bar, snack bar, cookie, candy, cereal, pudding, ice cream, frozen confectionary, chewing gum, drink mix, soda pop, liquid supplement, sauce, salad dressing, gravy, jelly, jam, spread, margarine, peanut butter, nut spread, frosting, and the like. In essence, can be used in any food, composition or supplement in which sugar is employed. Hence, the therapeutic agent and/or derivatives thereof can be used as a partial or full substitute for sugar.

Such food supplements, drinks and food items can include any other food ingredient including, for example, flour, oil, cream, butter, sugar, salt, spices and the like. In addition, the food supplements, drinks and food items can include vitamins and nutrients commonly found in other nutritional supplements.

Various routes of administration may be used in any of the methods described herein. In some embodiments of any of the methods described herein, the therapeutic agent can be administered by a variety of routes including oral, parenteral (including subcutaneous, intravenous, intramuscular, intraperitoneal, intraarticular, intraarterial, intrasynovial, or infusion techniques), rectal, dermal, transdermal, intrathoracic, intrapulmonary and intranasal (respiratory) routes.

Administration of the therapeutic agents in accordance may be in a single dose, in multiple doses, in a continuous or intermittent manner, depending, for example, upon the recipient's physiological condition, whether the purpose of the administration is therapeutic or prophylactic, and other factors known to skilled practitioners. The administration of the therapeutic agent may be essentially continuous over a pre-selected period of time or may be in a series of spaced doses. Both local and systemic administration is contemplated.

In certain of the methods described herein, the therapeutic agent is formulated for various forms of administration by any of the methods well known to the pharmaceutical arts. See, e.g., WO 2008/150477 and US 20090298112, incorporated herein in their entireties. The therapeutic agent may be administered, for example, at a dose of at least about 0.01 mg/kg to about 500 to 750 mg/kg, of at least about 0.01 mg/kg to about 300 to 500 mg/kg, at least about 0.1 mg/kg to about 200 to 400 mg/kg, at least about 1 mg/kg to about 25 mg/kg, or at least about 5 mg/kg to about 40 mg/kg, or at least about 1 mg/kg to 200 mg/kg, at least about 1 mg/kg to about 1000 mg/kg, at least about 200 mg/kg to about 1000 mg/kg, at least about 400 mg/kg to about 1000 mg/kg, or at least about 600 mg/kg to about 1000 mg/kg of body weight, although other dosages may provide beneficial results. The amount administered will vary depending on various factors including, but not limited to the disease, the weight, the physical condition, the health, the age of the mammal, whether prevention or treatment is to be achieved. Such factors can be readily determined by the clinician employing animal models or other test systems that are available in the art.

### Kits and Articles of Manufacture

As noted above, the present invention relates generally to diagnostic, prognostic and therapeutic methods, which are based, at least in part, on determining the sialylation state of one or more polysialic acid-glycoproteins in a biological fluid sample, such as a blood sample. In some embodiments, the methods use an agent to detect sialylated and unsialylated glycoprotein(s). In some embodiments, the methods use one or more antibodies to detect sialylated and unsialylated glycoprotein(s). In certain embodiments, the methods encompass administration of a therapeutic agent, e.g., as part of a sialic acid replacement therapy. Accordingly, the present invention includes kits and/or articles of manufacture for performing these methods as well as instructions for carrying out the methods of this invention such as collecting a blood sample, performing a screen, and/or analyzing the results, and/or administering an effective amount of a therapeutic agent as defined herein. These can be used alone or in combination with other suitable therapy.

Provided herein are kits and/or articles of manufacture comprising packaging material and at least one vial comprising an agent with the prescribed buffers and/or preservatives, optionally in an aqueous diluent, wherein the agent is used to determine sialylation state of one or more polysialic acid-glycoproteins in a blood sample. Also provided herein are kits and/or articles of manufacture comprising packaging material and at least one vial comprising a therapeutic agent with the prescribed buffers and/or preservatives, optionally in an aqueous diluent. Further provided herein are kits and/or articles of manufacture comprising packaging material and at least one vial comprising an agent with the prescribed buffers and/or preservatives, optionally in an aqueous diluent, wherein the agent is used to determine sialylation state of one or more polysialic acid-glycoproteins in a blood sample and at least one vial comprising a therapeutic agent with the prescribed buffers and/or preservatives, optionally in an aqueous diluent.

Provided herein are kits and/or articles of manufacture for use in diagnosing and/or assessing disease severity of a condition of sialic acid deficiency in an individual by detecting the sialylation state of one or more polysialic acid-glycoproteins in a blood sample from the individual. The kit and/or article of manufacture comprises, or alternatively consists essentially of, or yet further consists of, one or more suitable agent to determine the sialylation state of one or more polysialic acid-glycoproteins in a blood sample, and instructions for use thereof. In some embodiments, the kit and/or article of manufacture comprises, or alternatively consists essentially of, or yet further consists of, one or more suitable antibodies to determine the sialylation state of one or more polysialic acid-glycoproteins, and instructions for use thereof.

Further provided herein are kits and/or articles of manufacture for use in treating an individual suffering from a sialic acid deficiency, identifying an individual as suitable or not suitable for treatment, and/or selecting an individual for treatment based on the sialylation state of one or more polysialic acid-glycoproteins in a blood sample from the individual. The kit and/or article of manufacture comprises, or alternatively consists essentially of, or yet further consists of, one or more suitable agent(s) to determine the sialylation state of one or more polysialic acid-glycoproteins, one or more therapeutic agent(s) and instructions for use thereof. In some embodiments, the kit and/or article of manufacture comprises, or alternatively consists essentially of, or yet further consists of, one or more suitable antibodies, one or more therapeutic agent(s) and instructions for use thereof. In some embodiments, the sialylation state is hypo-sialylation, which indicates that the individual is suitable or selected for treatment. In some embodiments, the sialylation state is non-hypo-sialylation (e.g., normal sialylation), which indicates that the individual is not suitable for treatment.

Provided herein are also kits and/or articles of manufacture for use in monitoring responsiveness or lack of responsiveness to treatment in an individual and/or identifying an individual as suitable or not suitable to continue treatment with a therapeutic agent based on sialylation state of one or more polysialic acid-glycoproteins in a biological fluid sample from the individual. The kit and/or article of manufacture comprises, or alternatively consists essentially of, or yet further consists of, one or more suitable agent to determine sialylation state of one or more polysialic acid-glycoproteins, one or more therapeutic agent and instructions for use thereof. In some embodiments, the kit and/or article of manufacture comprises, or alternatively consists essentially of, or yet further consists of, one or more suitable antibody, one or more therapeutic agent and instructions for use thereof. In some embodiments, the sialylation state is hypo-sialylation, which indicates that the individual is non-responsive to current treatment, and might need optimization of treatment. In some embodiments, the sialylation state is non-hypo-sialylation (e.g., normal sialylation relative to a healthy control, hyper-sialylation relative to an earlier sample the same patient), which indicates that the individual is responsive to treatment.

In some embodiments, the biological fluid sample is a blood sample (e.g., serum sample). In some embodiments, the polysialic acid-glycoprotein is polysialic acid Neural Cell Adhesion Molecule (PSA-NCAM). In some embodiments, the sialic acid deficiency is Hereditary Inclusion Body Myopathy (HIBM).

The kits and/or articles of manufacture can include all or some of the positive controls, negative controls, reagents, and antibodies described herein for determining the sialylation state of one or more polysialic acid-glycoproteins in a biological fluid sample.

As amenable, these suggested kit and/or article of manufacture components may be packaged in a manner customary for use by those of skill in the art. For example, these suggested kit and/or article of manufacture components may be provided in solution or as a liquid dispersion or the like.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of non-critical parameters that could be changed or modified to yield essentially similar results.

### EXAMPLES

### Example 1

### Detection of Sialylated, Hypo-Sialylated And UNSIALYLATED PSA-NCAM in Serum Samples

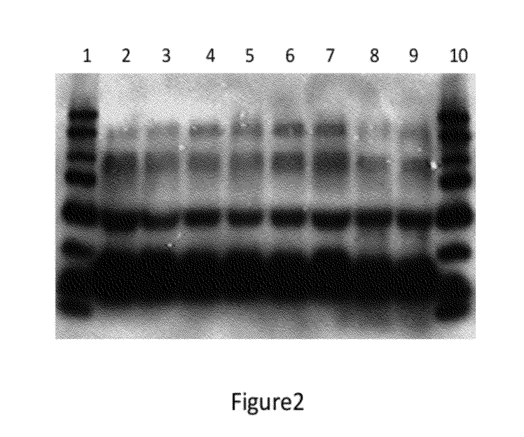

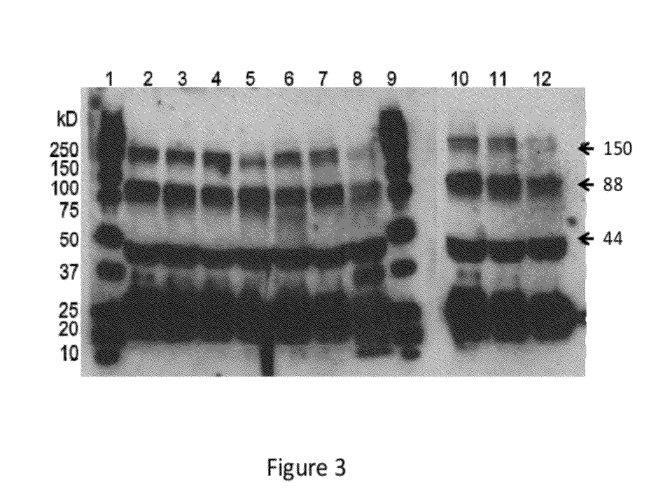

Serum samples from normal individuals, HIBM patients before treatment, patients suffering from myopathy but not HIBM, and a HIBM patient on ManNAc therapy for 2 years were purified to reduce serum proteins. The samples were subjected to SDS PAGE and stained with a monoclonal antibody to NCAM (RNL-1, SantaCruz Biotech, Inc., CA) capable of detecting sialylated and unsialylated forms of NCAM (FIGS. 2 and 3). As a control, serum samples from healthy individuals were prepared as described above and treated with sialidase to remove sialic acid.

For Western blotting, serum protein was extracted from 50 ul serum samples using Mem-PER® Eukaryotic Membrane Protein Extraction Reagent Kit (Thermo Scientific), then the hydrophilic (top) layer was cleaned and purified by SDS-PAGE Sample Prep Kit (Thermo Scientific). Final protein concentration was measured by BradfordUltra (Expedeon), and 390 ug protein (15 uL) loaded per lane for electrophoresis separation. Protein transfer was performed using Polyvinylidene Fluoride (PVDF) membrane, and confirmed by Ponceau stain. Primary (RNL-1, SantaCruz Biotech, Inc., CA) and secondary antibodies were applied, and detected using horseradish peroxidase (HRP) chemi-luminescence substrate (FIG. 3). Each lane represents single serum sample obtained by single blood draw.

The results show that some PSA-NCAM in serum was sialylated and PSA-NCAM sialylation varied in disease and treatment (FIGS. 2 and 3). The upper and middle bands detected by Western blot with the monoclonal antibody to PSA-NCAM were due to sialic acid containing forms, since they disappeared after treatment with sialidase (FIG. 2, Lanes 8-9; FIG. 2, Lane 8). These bands were fuzzy and decreased in HIBM patients (FIG. 2, Lanes 2-3; FIG. 2, Lane 5), and improved after sialic acid replacement treatment (FIG. 3, Lane 7). They were not abnormal in patients with muscle disease but not HIBM (FIG. 3, Lanes 4-5; FIG. 3, Lane 6).

As shown in FIG. 3, Western blot analysis of PSA-NCAM was performed on control serum (non-myopathic) (FIG. 3, lanes 2-4 and 10-11), myopathic serum (non-HIBM) (FIG. 3, lane 6), and HIBM serum samples (FIG. 3, lanes 5 and 12). For desialylated control sample, serum protein from healthy non-myopathic individuals was treated with Vibrio cholerae neuroaminidase (Sigma) (FIG. 3, lane 8). HIBM patients who were routinely ingesting N-Acetylmannosamine (ManNAc) were analyzed as well (FIG. 3, lane 7). The two HIBM patient serum samples (lanes 5 and 12) showed an intermediate signal between de-sialylated (lane 8) and control serum (lanes 2-4 and 10-11) at ˜150 kD band marked by arrow. The ManNAc self-treated HIBM patient (lane 7) showed an improved signal at ˜150 kD band.

### Example 2

### Determining Responsiveness to Sialic Acid Replacement Therapy in HIBM Patients

Individuals diagnosed with HIBM are treated with sialic acid replacement therapy (e.g., sialic acid, ManNAc) and responsiveness to treatment is assessed in serum samples of these individuals by detecting polysialylated and hypo-sialylated PSA-NCAM as described in Examples 1-2. A change in sialylation status of PSA-NCAM in the biological fluid sample is indicative for the responsiveness of the individual. A decrease in hypo-sialylation or non-hypo-sialylation may indicate that the individual is responsive to treatment. If an individual is responsive, treatment will be continued. If the individual is not responsive, treatment may not be continued or the dosage of the agent is increased and/or further modified.

### Example 3

### Detection of PSA-NCAM Sialylation States in Normal Human Serum

Experiments were performed to determine whether PSA-NCAM can be detected in human serum using well-defined mouse monoclonal antibodies raised against human PSA-NCAM (123C3). Human serum was purified by Aurum serum protein kit (Bio-Rad) to remove albumin and IgG which can interfere with SDS-PAGE analysis. Raw serum was diluted 4 times with protein binding buffer (supplied in the kit) and applied to the column. The albumin/IgG depleted serum was eluted by centrifugation at 10,000 g for 20 seconds and protein concentration was determined by modified Lowry assay (Bio-Rad DC protein assay).

Purified serum was incubated in neuraminidase from Vibrio cholera (Sigma) at 37° C. for 4 hours in order to cleave sialic acid (SA) from serum polysialic acid-glycoproteins, including PSA-NCAM. Digestion was carried out at a concentration of 1 m unit enzyme per 3 μg of protein in 20 mM Tris buffer, pH 5.8. A negative control was included in the experiment under the same condition but without adding the neuraminidase. At the end of incubation, the mixture was cleaned up with micro bio-spin P30 columns (BioRad).

Proteins were analyzed on 10% denaturing Tris-glycine SDS-PAGE gel (Invitrogen) under reducing condition. At the end of the electrophoresis, proteins on the gel were transferred to polyvinylidene fluoride (PVDF) membranes using Mini Trans-Blot electrophoretic transfer cell (BioRad) in 1× Towbin buffer (25 mM Tris, 192 mM glycine) with 0.01% SDS. Transfer was conducted at 4° C. for 12 hours at constant voltage of 30V.

For Western blot, PVDF membrane was incubated with protein-free T20 blocking buffer (Thermo Scientific). The membrane was subsequently probed with primary antibody diluted in TBST buffer (20 mM Tris pH7.4, 150 mM NaCl, 0.1% Tween 20 and 0.5% gelatin) at 4° C. for 12 hours and then washed 6×10 minutes in TBST buffer. In the experiment, the mouse monoclonal antibody 123C3 (Santa Cruz Biotechnology) was used at 1:100 concentration (final concentration 2 μg per ml). This antibody was raised against membrane preparation of human small cell lung carcinoma and recognized multiple PSA-NCAM isoforms, including PSA-NCAM. After washing, the blot was incubated with anti-mouse IgG-HRP conjugated secondary antibody (BioRad) at room temperature for 1 hour and then washed 6×10 minutes in TBST buffer. The secondary antibody was diluted 1:5000 in TBST buffer. The blot was developed with ECL Western Blotting Substrate (Thermo Scientific) and the images were exposed by Autorad film (GeneMate) and developed by automatic X-ray film processor.

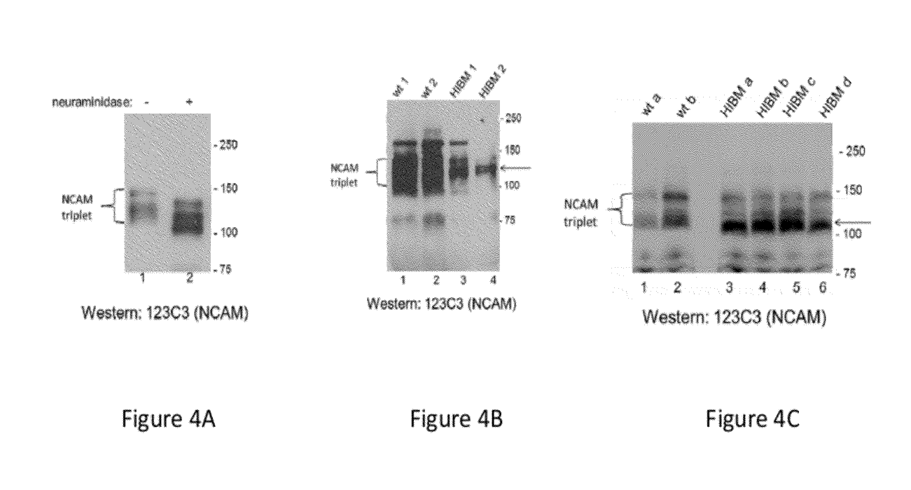

As shown by the Western blot in FIG. 4A, PSA-NCAM appeared as a triplet in normal human serum ranging from 110-130 kDa without neuraminidase treatment (lane 1). The triplet could represent different PSA-NCAM isoforms and/or PSA-NCAM with different post-translational modifications. When treated with neuraminidase to remove SA, the molecular weight of the PSA-NCAM triplet was reduced by an estimated 10-15 kDa (FIG. 4A, lane 2). This result indicated that serum PSA-NCAM is sialylated and contains a fair amount of SA.

### Example 4

### Detection of PSA-NCAM Sialylation States in HIBM Human Serum

Experiments were then performed to analyze PSA-NCAM in serum of HIBM patients and to determine whether the sialylation pattern is different from normal human controls. Human serum was purified by a two-steps protocol to enrich PSA-NCAM. Raw sera from both normal human and HIBM patient were extracted by Mem-PER® membrane protein extraction kit (Thermo Scientific) according to the manufacturer protocol. Serum was first mixed with a detergent and then a second detergent was added to solubilize membrane proteins. After centrifugation at 10,000 g for 3 minutes, the mixture was incubated at 37° C. to separate hydrophobic proteins from hydrophilic proteins through phase partitioning. The top hydrophilic layer was then further purified by SDS-PAGE sample prep kit (Thermo Scientific) to remove detergents and other contaminants that interfere with SDS-PAGE analysis. Proteins were analyzed on 10% Tris-glycine gel and Western blots were performed as described above.

As shown in FIG. 4B, Western blot demonstrated that PSA-NCAM was present as a triplet in two normal human sera (lanes 1 and 2). On the other hand, the PSA-NCAM triplet was less distinguishable in the HIBM sera (lanes 3 and 4). In one of the HIBM patients (HIBM 2), the top bands of the PSA-NCAM triplet were substantially decreased in intensity and PSA-NCAM appeared as a single species of lower molecular weight (lane 4, arrow). Similar findings were observed in the replicate samples using a different PSA-NCAM antibody, ANC7C7 (Santa Cruz Biotechnology) which was raised against extracellular domains 1-4 of recombinant human PSA-NCAM.

Overall, the sialylated PSA-NCAM bands were altered in the HIBM patients and did not represent the normal pattern. For instance, it appeared that the lower molecular weight species of the PSA-NCAM triplet was preferentially retained in the HIBM serum. This result suggests that PSA-NCAM sialylation was changed in the HIBM serum. For development of a serum biomarker, these experiments show that it is possible to assay PSA-NCAM sialylation in serum by Western blot, based, for example, on the shift in molecular weight of PSA-NCAM or a change in the PSA-NCAM triplet pattern in the HIBM patients. PSA-NCAM in serum is thus a useful biomarker to assess sialylation in HIBM patients.

### Example 5

### Detection of PSA-NCAM Sialylation States in HIBM Human Serum

Further experiments were performed to analyze PSA-NCAM sialylation state in the serum of HIBM patients and to determine whether the sialylation differs from normal human controls. Human serum was diluted 4 times in protein binding buffer (Aurum serum protein kit, BioRad) and approximately 380 μg of diluted raw serum was loaded on 6% Tris-glycine gel. Western blots were performed as described above using 123C3 (PSA-NCAM) antibody.

As shown in FIG. 4C, PSA-NCAM was detected by the 123C3 antibody as a triplet in two normal human sera (lanes 1 and 2). As shown in lanes 3-6, the lower band of the PSA-NCAM triplet appeared to migrate slightly faster in the sera of four different HIBM patients (arrow), indicating that it has a lower molecular weight. The lower band also seemed to be sharper and the intensity of the signal was stronger relative to normal (healthy) controls.

Similar to above, the sialylated PSA-NCAM bands were altered in the HIBM patients and did not represent the normal PSA-NCAM pattern on Western blot. It appeared that the lower band of the PSA-NCAM triplet migrated faster in the HIBM serum. This result suggests that the PSA-NCAM sialylation was changed in the HIBM serum. It is thus possible to assay PSA-NCAM sialylation in serum, for example, by Western blot based on the shift of MW or change of PSA-NCAM triplet pattern in HIBM patients.

### Example 6

### Detection of PSA-NCAM Sialylation States in Normal Human and HIBM Serum using Polyclonal Antibody

Experiments were performed to investigate the pattern of serum PSA-NCAM sialylation between normal controls and HIBM patients using a polyclonal antibody raised against human PSA-NCAM. Human serum was purified by a two-steps protocol to enrich PSA-NCAM. Raw sera from both normal human and HIBM patient were extracted by Mem-PER® membrane protein extraction kit (Thermo Scientific) according to the manufacturer's protocol. Serum was first mixed with a detergent and then a second detergent was added to solubilize membrane proteins. After centrifugation at 10,000 g for 3 minutes, the mixture was incubated at 37° C. to separate hydrophobic proteins from hydrophilic proteins through phase partitioning. The top hydrophilic layer was then further purified by SDS-PAGE sample prep kit (Thermo Scientific) to remove detergents and other contaminants that interfere with SDS-PAGE analysis.

Purified serum was incubated with Vibrio cholera neuraminidase (Sigma) at 37° C. for 4 hours in order to cleave sialic acid (SA) from serum polysialic acid-glycoproteins, including PSA-NCAM. Digestion was carried out at a concentration of 0.35 m unit enzyme per μl of serum. Negative control was included in the experiment under the same condition but without adding the neuraminidase.

Proteins were analyzed on a 10% Mini-Protean precast gel (BioRad) under reducing condition with 5% 2-mercaptoethanol. Final protein concentration was measured by Bradford Ultra (Expedeon). Proteins on the gel were electrophoretically transferred to polyvinylidene fluoride (PVDF) membranes using Mini Trans-Blot electrophoretic transfer cell (BioRad) in 1× Towbin buffer (25 mM Tris, 192 mM glycine) with 0.01% SDS. Transfer was conducted at 4° C. for 12 hours at constant voltage of 30V.

For Western blot, the PVDF membrane was placed in 1:10 diluted solution of Ponceau S Stain (Amresco) in order to visualize the bands. The stain was washed off immediately using TBST (25 mM Tris, pH 7.4, 0.1% Tween 20) followed by blocking in 5% Blotting Grade Blocker Non-Fat Dry Milk (BioRad) in TBST (blocking) buffer for 1 hour at room temperature. The membrane was then probed with primary antibody in diluted in blocking buffer. In this experiment, an affinity-purified goat polyclonal antibody C-20 (Santa Cruz Biotechnology) was used at a final concentration of 0.02 μg per ml. This antibody was raised against the C-terminus of human PSA-NCAM. The membrane was washed in TBST for 25 minutes at room temperature and then every five minutes for 4 times. Finally, the membrane was probed with goat anti-mouse IgG-HRP secondary antibody (abcam) diluted in blocking buffer at a final concentration of 0.2 μg per ml for 1 hour at room temperature at 25 rpm, and washed again as described above. To develop the signal, the membrane was incubated in SuperSignal® West Dura Extended Duration Substrate (Thermo Scientific) for 5 minutes and subsequently developed by automatic X-ray film processor following 30 sec film exposure.

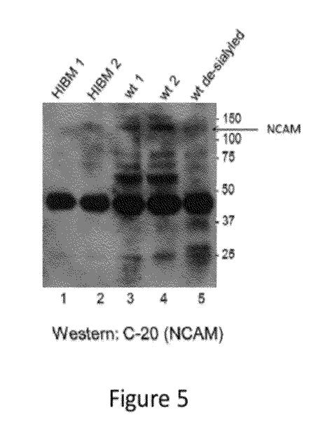

As shown in the Western blot of FIG. 5, a pattern of PSA-NCAM bands between 60 kDa and 150 kDa was observed in normal human (lanes 3 and 4). The major PSA-NCAM band had a molecular weight of ˜130 kDa (arrow). Another major PSA-NCAM band of low molecular weight was detected at around 60 kDa. The intensity of both the 130 kDa and 60 kDa bands was significantly reduced in the sera of HIBM patients (lanes 1 and 2). The major band below 50 kDa was human IgG heavy chain cross-reacted with the secondary antibody and its pattern and intensity did not change in either normal human or HIBM patients. When normal serum was treated with neuraminidase to remove SA, both the 130 kDa and 60 kDa bands were significantly reduced in intensity (lane 5). This result suggested that de-sialylation might lead to decreased levels or stability of PSA-NCAM proteins.

This study confirms that multiple PSA-NCAM of different molecular weight can be detected by the C-20 polyclonal antibody in normal human serum. In HIBM patients, these PSA-NCAM bands were significantly reduced or absent. Since de-sialylation of serum proteins can affect PSA-NCAM stability, the loss of PSA-NCAM signal in HIBM serum could be an indication of hypo-sialylation. PSA-NCAM in serum is thus a useful biomarker to assess sialylation in HIBM patients. These data suggest that PSA-NCAM signal in serum of HIBM patients is reduced compared to normal controls. For development of a serum biomarker, it is possible to assay PSA-NCAM in serum by Western blot or quantitatively by ELISA based on the presence of absence of these PSA-NCAM bands in the patients.

1. A method for diagnosing a condition of sialic acid deficiency in a subject comprising determining the sialylation state of a polysialic acid-glycoprotein in a blood sample from the subject, and diagnosing the subject as sialic acid deficiency if the sialylation state is less than a pre-determined level.

2. The method of claim 1, wherein the sialylation state of the polysialic acid-glycoprotein is determined based on the molecular weight of the polysialic acid-glycoprotein.

3. The method of claim 1, wherein the sialylation state is less than a pre-determined level if the molecular weight of the polysialic acid-glycoprotein in the blood sample is less than a pre-determined molecular weight.

4. The method of claim 1, wherein the polysialic acid-glycoprotein is expressed in muscle tissue.

5. The method of claim 1, wherein the polysialic acid-glycoprotein comprises a polysialic acid polymer.

6. The method of claim 1, wherein the polysialic acid-glycoprotein comprises a polysialic acid polymer including from at least about 5 sialic acid residues to about 50 sialic acid residues.

7. The method of claim 1, wherein the polysialic acid-glycoprotein is polysialic acid-neural cell adhesion molecule (PSA-NCAM).

8. The method of claim 1, wherein the blood sample is a serum or plasma sample.

9. The method of claim 1, wherein the pre-determined level is a level determined based on a population without sialic acid deficiency.

10. The method of claim 1 further comprising recommending the subject for treatment of sialic acid deficiency.

11. The method of claim 1 further comprising determining the level of sialic acid deficiency based on the level of decrease of the sialylation state from the pre-determined level.

12. The method of claim 1, wherein the sialic acid deficiency is Hereditary Inclusion Body Myopathy (HIBM), Nonaka myopathy, or Distal Myopathy with Rimmed Vacuoles (DMRV).

13. A method for monitoring responsiveness or efficacy of a treatment to a subject suffering from sialic acid deficiency comprising determining the sialylation state of a polysialic acid-glycoprotein in a blood sample from the subject, wherein an increase of the sialylation state of the polysialic acid-glycoprotein is indicative of responsiveness or efficacy of the treatment.

14. The method of claim 13 further comprising determining future treatment regimen based on the sialylation state of the polysialic acid-glycoprotein in the blood sample.

15. A method for determining whether a subject is suitable for a sialic acid replacement therapy comprising
determining the sialylation state of a polysialic acid-glycoprotein in a blood sample from the subject,
wherein a subject is suitable for a sialic acid replacement therapy if the sialylation state of the polysialic acid-glycoprotein is less than a predetermined level and wherein a subject is not suitable for a sialic acid replacement therapy if the sialylation state of the polysialic acid-glycoprotein is equal or higher than the pre-determined level.

16. A method for treating a subject comprising
determining the sialylation state of a polysialic acid-glycoprotein in a blood sample from the subject,
administering a sialic acid replacement therapy to the subject if the sialylation state of the polysialic acid-glycoprotein is less than a pre-determined level.

17. A collection of molecular weight data comprising the molecular weight of a polysialic acid-glycoprotein in a blood sample from a testing subject.

18. The collection of claim 17 further comprises the molecular weight of the polysialic acid-glycoprotein in a blood sample from a control subject.

19. A method for providing data comprising
determining the sialylation state of a polysialic acid-glycoprotein in a blood sample from a subject,
providing the information of the sialylation state to a healthcare provider for diagnosing or treatment of the subject.

20. The method of claim 19 further comprising receiving the blood sample from the healthcare provider.

21. A method of assaying the sialylation state of a polysialic acid-glycoprotein in a subject comprising
obtaining or receiving a blood sample of the subject, and
determining the sialylation state of a polysialic acid-glycoprotein in the blood sample.

22. The method of claim 21, wherein the subject has, is at risk of having, or is suspected of having a condition of sialic acid deficiency.
